(19) United States
(12) Patent Application Publication
(10) Pub. No.: US 2002/0188963 A1
(43) Pub. Date: Dec. 12, 2002

(54) Pluripotent embryonic stem cells and methods of obtaining them

(76) Inventor: Jeanne F. Loring, Foster City, CA (US)

Correspondence Address: Karen R. Zachow, Morrison & Foerster LLP, 755 Page Mill Road, Palo Alto, CA, 94304-1018, US

(21) Appl. No.: 10/165,765

(22) Filed: Jun. 6, 2002

Related U.S. Application Data
(63) Continuation of application No. 09/199,703, filed on Nov. 24, 1998.
(60) Provisional application No. 60/066,890, filed on Nov. 25, 1997.

(51) Int. Cl. A01K 67/027; C12N 5/08; C12N 5/06

(52) U.S. Cl. 800/14; 435/325; 435/366; 435/353

(57) ABSTRACT

The present invention provides an isolated population of non-mouse embryonic stem (ES) cells and methods of obtaining these ES cells. In one aspect, the target ES cells are obtained by co-culturing embryo cells from a target animal with non-target ES cells, such as mouse ES cells. In one embodiment, rat ES cells are isolated from the co-culture using positive or negative selectable markers.

The invention also includes genetically modified non-mouse ES cells. Chimeric embryos and animals containing isolated populations of the ES cells or genetically modified ES cells are also provided. In one embodiment, the genetic modification comprises introduction of a transgene. In another embodiment, the genetic modification comprises disruption of the function of one or more genes.

### CROSS-REFERENCE TO RELATED APPLICATION

The present application claims the priority benefit of U.S. provisional patent application No. 60/066,890 filed Nov. 25, 1997, pending. The aforementioned provisional application is hereby incorporated herein by reference in its entirety.

### TECHNICAL FIELD

This invention is in the field of molecular biology and medicine. More specifically, it relates to novel non-mouse embryonic stem (ES) cells and methods of obtaining these non-mouse ES cells.

### BACKGROUND

Genetically modified laboratory animals are widely used in drug development and as model systems of human disease. Many of these transgenic animals, especially loss-of-function mutants, are mouse models that have been generated by using mouse embryonic stem (ES) cells. Briefly, gene targeting in ES cells uses the phenomenon of homologous recombination to disrupt or knock-out the function of a particular gene. (See, U.S. Pat. Nos. 5,464,764; 5,487,992; 5,631,153 and 5,627,059 for techniques relating to mouse embryonic stem cells). There is a rapidly growing number of mouse mutants that have been created by inactivation of genes in ES cells. The mutant mice are produced to understand the function of known genes in vivo, to discover new genes and to create animal models of human diseases. (see, e.g., Chisaka et al. (1992) 355:516-520; Joyner et al. (1992) in POSTIMPLANTATION DEVELOPMENT IN THE MOUSE (Chadwick and Marsh, eds., John Wiley & Sons, United Kingdom) pp:277-297; Dorin et al. (1992) Nature 359:211-215).

Although the development of mouse ES cells inaugurated a new era for mouse genetics, non-mouse ES cells have proven difficult to isolate. Putative cell lines have been derived from blastocysts of hamster, pig and sheep. (see, Doetschman et al. (1988) Develop. Biol. 127:224-227; Notarianni et al. (1990) J. Reprod. Fertil. Suppl. 41:51-56; Notarianni et al. (1991) J. Reprod. Fertil. Suppl. 43:255-60; WO 94/26884). It is not clear, however, if these cells are truly pluripotent ES cells. (see, e.g, Gardner and Brook (1997) Int. J. Dev. Biol. 41:235-243).

Gerhart et al. report isolation of human gonad-precursor cells from aborted fetuses. (See, Beardsley (1998) Scientific American, on line). When these cells are implanted into mice with no functioning immune system, they apparently give rise to tumors containing various cell types. However, it is not clear that these isolated human cells are pluripotent ES cells.

Of particular interest in the transgenic field are rat pluripotent cells. Transgenic rats offer several advantages over mouse models. First, rats are larger, which makes surgical procedures possible, and they provide a larger amount of material for biochemical analysis. A second advantage of rat models is that a large existing database of information has been generated for rat models, especially in the areas of neurodegenerative disease, cardiovascular research and diabetes. No comparable database exists for-mice.

Rats are also the preferred animal model for drug development-assays. Transgenic rats allow sophisticated physiological measurements that are not possible in transgenic mice. For example, because of their size, rats harboring human transgenes can offer primate-specific analyses in a non-primate experimental system. In some cases, the physiology of rats allows production of transgenic animals whose phenotypes are useful for human disease models, while mice carrying the same transgene have no disease phenotype. For example, a transgenic model of HLA-B27-associated human autoimmune disorders was first attempted in mice, but no pathology developed (Taurog et al. (1988) J. Immunol. 141:4020-30). Transgenic HLA-B27 rats, however, have pathologies that closely resemble the human disease (Hammer et al. (1990) Cell 63:1099-1112). Thus, rat ES cells could be used to exploit all of the genetic manipulations now possible only in mice. If rat ES cell technology were available, null mutations could be introduced into the rat homologues of human genes, and in combination with the human transgenes, would provide a “humanized” animal that could replace the primate for many studies.

Despite continued attempts, no one has yet succeeded in deriving rat ES cells. lannoccone et al. (1994) Develop Biol. 163:288-292 (see, also WO 95/06716) originally reported isolation of rat embryonic stem cells, but recently retracted this publication after discovering that the cell lines were contaminated with mouse ES cells. (Brenin et al. (1997) Develop Biol. in press; Brenin et al. (1996) In: PHARM. CEREBRAL ISCHEMIA (ed. Krieglstein, Medpharm Scienfitic Publishers, Stuttgart) pp:555-572). The present invention, herefore, provides the first isolated population of rat ES cells.

It seems that the initial success of culturing the mouse cells may have been due partly to a fortuitous choice of mouse strain; the 129 strain of mouse had been used for earlier teratocarcinoma work because of its tendency toward testicular germ cell tumors. (see, Evans & Kaufman(198-1) Nature 292:154-156; Martin (1981) Proc. Nat'l Acad. Sci. USA 78:7634-7638).I believe that pluripotent cells in the blastocyst inner cell mass of this particular strain have an inherent ability to adapt to tissue culture. Other strains, such as the C57BL/6 mouse, have proven to be more difficult for derivation of ES cell lines, and only recently was a germ line competent C57BL/6 ES cell line reported (Ledermann et al. (1991) Exp. Cell Res. 197:254-258). There is no rat strain that has a tendency to develop testicular teratocarcinomas, and the lack of such a strain is likely one of the reasons why the derivation of the first rat ES cell lines has been more difficult. Also, in contrast to the mouse, experimentally induced teratocarcinomas in the rat usually derive from the yolk sac rather than from germ cells; these cells are not pluripotent, but rather retain features of the yolk sac endoderm. This characteristic may lead to the rapid dilution in cultures from blastocysts of some rat strains of ES-like cells with cells that morphologically resemble endoderm cells.

The present invention provides the first pluripotent rat ES cells. In addition, the invention provides a method which can be universally applied to the generation of ES cells from all species.

### SUMMARY OF THE INVENTION

The present invention includes an isolated population of non-mouse embryonic stem cells. In one embodiment, the cells are rat ES cells.

In another aspect, the invention provides a method of obtaining embryonic stem cells from a target species, the method comprising: (a) co-culturing cells obtained from an embryo of the target species with non-target embryonic stem cells under conditions which favor growth of embryonic stem (ES) cells from the target species: and (b) isolating the ES cells from the target species. The non-target embryonic stem cells used in the co-culture are from a species other than the target species, such as mouse. The target ES cells can be derived from inner cell masses (ICMs) or can be primordial germ cells (PGCs).

In another embodiment, the non-target embryonic stem cells of used in the co-culture lack a positive selection marker. The positive selection marker can be a gene encoding antibiotic resistance or a gene encoding HPRT resistance (HPRT). In yet another embodiment, the embryonic stem cells used in the co-culture carry a negative selection marker, for example HPRT or herpes simplex virus thymidine kinase (HSV-tk). Optionally, the embryo cells can be cultured on a feeder layer of cells. In one embodiment the feeder layer is SNL 76/7 cells. In another embodiment, the non-target ES cells used in the co-culture methods are mitotically inactivated.

In yet another aspect, the present invention includes genetically modified non-mouse ES cells. In one embodiment, the genetic modification comprises insertion of a transgene. In another embodiment, the genetic modification comprises disrupting the function of one or more genes.

In a further aspect, the invention includes a chimeric embryo containing the isolated population of ES cells or genetically modified ES cells. In one embodiment, the chimeric embryo contains ES cells that have been genetically modified to include a transgene. In another embodiment, the chimeric embryo contains ES cells that have genetically modified to disrupt the function of one or more genes.

In another aspect, the invention includes an animal containing cells arising from an isolated population of target ES cells. In one embodiment, the animal contains cells arising from rat ES cells that have been genetically modified to include a transgene. In another embodiment, the animal contains cells arising from rat ES cells that have genetically modified to disrupt the function of one or more genes.

As will become apparent, preferred features and characteristics of one aspect of the invention are applicable to any other aspect of the invention.

### BRIEF DESCRIPTION OF THE DRAWINGS

FIG. 1, panels A through D, are half-tone reproductions of photographs showing rat blastocysts.

FIG. 1A shows 400× magnification, Nomarski optics of normal rat blastocysts from the Brown Norway strain;

FIG. 1B shows 400× magnification, Nomarski optics of rat blastocysts from the Fischer 344 strain where implantation was delayed 10 days.

FIG. 1C shows 200×magnification, phase contrast of a group of delayed rat blastocysts from the Long Evans strain where implantation was delayed 8 days.

FIG. 1D blastocysts attached after 3 days in culture on SNL 76/7 fibroblast feeder layer. The inner cell mass (ICM) is visible at the center of the cultures.

FIG. 2, panels A through C, are half-tone reproductions of photographs showing cultures derived from the ICMs of rat blastocysts.

FIGS. 2A and 2B show colonies in secondary culture of FRDB-1 cell line. The morphology of the colonies resembles mouse ES cells at this stage of derivation.

FIG. 2C shows a third passage of cell line BNRB-1. The colonies have a more epithelial morphology, typical of endoderm.

FIGS. 2A-C are 200× magnification, phase contrast microscopy.

FIG. 3; panels A through E are half-tone reproductions of 200× magnification of cultured blastocysts.

FIGS. 3A and 3C show alkaline phosphatase (AP) staining of inner cell mass cultured rat and mouse blastocysts, respectively.

FIG. 3B shows rat blastocyst-derived cells (line BNRB-1) stained with AP;

FIG. 3D shows mouse ES cells stained with AP.

FIG. 3E shows AP positive cells within a colony derived from 9 day Sprague-Dawley embryo. These cells may be derived from primordial germ cells.

FIG. 4A shows a phase contrast view of Long Evans rat blastocyst inner cell mass (ICM) cells cultured for 3 days before staining;

FIG. 4B shows fluorescence microscopy of the 3 day cultured ICM cells stained with anti SSEA-1 antibody.

FIG. 4C shows mouse ES cells stained with anti SSEA-1. SSEA-1 is heterogeneously expressed.

FIG. 4D shows colonies from the BNRB-1 cell line stained with SSEA-1. The rat line is also heterogeneous for SSEA-1 labeling.

FIG. 5A shows a cystic structure, similar to mouse ES cell-derived simple embryoid body, formed by BRNB-1 cell line after culture in suspension.

FIG. 5B shows morphological changes in the BNRB-1 cell line grown on a rat embryonic fibroblast feeder layer;

FIG. 5C shows morphological changes in mouse ES cells grown on a rat embryonic fibroblast feeder layer.

FIG. 6, panels A through D, are half-tone reproductions of 10× magnification, phase contrast photographs showing the morphology of cell co-cultures of rat blastocysts and mouse ES cells.

FIG. 6A depicts ICM from a delayed Long Evans rat blastocyst after three days of culture on mouse AB-1 ES cell feeder layer. The ICM cells look like mouse ES cells, and no differentiation is apparent.

FIG. 6B shows second passage of LE rat ICM by mechanical dissociation. Loosely adherent spherical cells are present on the edges of the explant.

FIG. 6C shows LE rat ICM after 7 passages. The ICM explant looks like mouse ES cells, but spherical cells are much more abundant. These fast-growing spherical cells are believed to be endoderm. After several more passages of the cells having ES morphology, there were very few alkaline phosphatase (AP)-positive cells remaining.

FIG. 6D shows AP staining of an early passage rat ICM. The spherical endoderm-like cells are negative.

FIG. 7, panels A through C, are half-tone reproductions of photographs of rat primordial germ cells (PGC) in culture.

FIG. 7A shows a 10× magnification phase contrast photograph of PGC cultures derived by dissociating the hindgut tissue and allantois from a 10.5 day LE rat embryo and co-culturing with mouse ES cells for two passages. After two passages (mouse ES cell confluence), the mouse ES cells were removed by negative selection. The remaining rat cells were passaged once more and the culture examined at 17 days. Surviving rat cells are apparent.

FIG. 7B shows a 20× magnification the cells in FIG. 7A.

FIG. 7C shows the same colony as shown in FIGS. 7A and` 7B stained for alkaline phosphatase(AP).after 21 days in culture. The morphology and staining indicates that these cells have properties of ES cells.

### MODES FOR CARRYING OUT THE INVENTION

Throughout this application, various publications, patents, and published patent applications are referred to by an identifying citation. The disclosures of these publications, patents, and published patent specifications referenced in this application are hereby incorporated by reference into the present disclosure to more fully describe the state of the art to which this invention pertains.

The present invention provides pluripotent embryonic stem (ES) cells. The ES cells are typically not mouse and, although isolated rat ES cells are exemplified herein, the claimed methods can be applied to any species. In general, the invention provides substantially purified populations of non-mouse, for example rat, ES cells. The methods of producing ES cells involve co-culturing embryo cells from a target species (e.g., inner cell masses (ICMs) from blastocysts or primordial germ cells (PGCs)) with non-target ES cells. The non-target ES cells can be from any species, for instance mouse. Preferably, the non-target ES cells contain a negative selection marker.

The practice of the present invention will employ, unless otherwise indicated, conventional techniques of molecular biology, microbiology, cell biology and recombinant DNA, which are within the skill of the art. See, e.g., Sambrook, Fritsch, and Maniatis, MOLECULAR CLONING: A LABORATORY MANUAL, 2nd edition (1989); CURRENT PROTOCOLS IN MOLECULAR BIOLOGY, (F. M. Ausubel et al. eds., 1987); the series METHODS IN ENZYMOLOGY (Academic Press, INC.), PCR 2: A PRACTICAL APPROACH (M. J. McPherson, B. D. Hames and (G. R. Taylor eds., 1995); ANIMAL CELL CULTURE (R. I. Freshney. Ed., 1987); and ANTIBODIES: A LABORATORY MANUAL (Harlow et al. eds., 1987).

Definitions

As used herein, certain terms will have specific meanings.

The terms “embryonic stem cell” or “ES cell” are used to refer to cells of the early embryo that can give rise to all differentiated cells, including germ line cells. Although not yet isolated from every species, all animals are believed to have ES cells. Mouse embryonic stem cells (the most well-characterized ES cell) are derived from the pluripotent inner cell mass of blastocysts, and their pluripotence can be maintained by an appropriate culture environment. Mouse ES cells lines have been established in culture using feeder cells such as irradiated fibroblasts or cultured in medium conditioned by established teratocarcinoma stem cell lines. Some mouse ES cells can also be propagated without a feeder cell layer in the presence of differentiation inhibiting activity (DIA) or leukemia inhibitory factor (LIF) which prevent spontaneous differentiation of cells in culture.

The terms “non-mouse” and “target” ES cells refer to cells derived from any animal other than mouse. Preferred non-mouse or target cells are rat. The term “non-target” ES cells refers to cells derived from any animal other than the target species. Preferred non-target ES cells are mouse.

When they are transplanted to host blastocysts, ES cells contribute to formation of chimeric animals, and if the germ cells of a chimera are ES cell-derived, the offspring of the chimera carry the genome of the ES cells (“germ-line transmission”). “Known” ES cells are those which have been shown to be pluripotent as determined by assays and methods known in the art and described herein. Pluripotent cell lines are not limited to blastocyst-derived lines; recently, a cell line that possesses at least the in vitro pluripotence of ES cells was derived from mouse primordial germ cells (see, Matsui et al. (1992) Cell 70:841-847; Matsui and Hum (1997) Cell 10(1):63-8; Buehr, M. (1997) Exp. Cell Res. 232:194-207).

ES cells can be easily manipulated. They are useful as models for studies of cellular differentiation, presumably because the factors they produce and secrete are important for the control of early embryonic development in vivo Genetically manipulated ES cells harboring foreign DNA can be used to generate lines of true-breeding transgenic animals. Methods of genetic manipulation are known to those skilled in the art.

An “isolated” or “purified” population of cells is substantially free of cells and materials with which it is associated in nature. By substantially free or substantially purified is meant at least 50% of the population are ES cells, preferably at least 70%, more preferably-at least 80%, and even more preferably at least 90% free of non-pluripotent ES cells with which they are associated in nature.

A “cell line” or “cell culture” denotes higher eukaryotic cells grown or maintained in vitro. It is understood that the descendants of a cell may not be completely identical in morphology, genotype or phenotype to the parent cell.

The term “embryo” refers to tissue obtained from any stage of an animal's development prior to birth. In the course of mammalian development, for instance, the fertilized egg cleaves to form a mulberry-shaped cluster of cells called the “morula.” Between the 8- and 16-cell stage, the morula transforms into a “blastocyst” —a nearly spherical, fluid-filled structure. The outer cells of the blastocyst are “trophectoderm” cells and give rise to the placenta and other extraembryonic structures. The embryo itself is derived from the “inner cell mass” or “ICM,” an accumulation of cells at one pole of the blastocyst. Other embryonic tissue sources include delayed blastocysts and primordial germ cells.

As described herein, the methods developed by the present inventor are equally applicable to all species. The term “target animal” or “target species” refers to the species from which the isolated ES cells of the present invention are derived. Suitable species include but are not limited to, rat, human, bovine and sheep. The term “selectable marker” refers to a gene whose expression allows one to identify cells that have been transformed or transfected with a vector containing the marker gene. Selectable markers can be “positive” or “negative” and dominant or recessive. A “positive selection marker” refers to a gene encoding a product that enables only the cells that carry the gene to survive and/or grow under certain conditions. For example, plant and animal cells that express the introduced antibiotic resistant genes. Non-limiting examples of suitable antibiotic resistant genes are neomycin resistance (Neo) which confers resistance to the compound G418, hygromycin resistance and puromycin resistance. Another positive selection marker is hypoxanthine phosphoribosyl transferase (HPRT). Cells that carry the HPRT gene grow in HAT (amniopterin, hypoxanthine, thymidine) medium, while cells that are HPRT-negative (HPRT) die in HAT medium.

Similarly, the term “negative selection marker” refers to a gene encoding a product that can be induced to selectively kill the cells that carry the gene. Non-limiting examples of negative selection markers include herpes simplex virus thymidine kinase (HSV-tk) and HPRT. Cells carrying the HSV-tk gene are killed when gancyclovir or FIAU (1(1,2-deoxy-2-fluoro-β-D-rabinofuranosyl)-5-iodouracil) is added and cells carrying mammalian tk are killed using 5-bromodeoxyuridine (5BdU). Cells carrying the HPRT marker can be selectively killed with 6-thioguanine (6TG). Other examples of selectable markers (both positive and negative) will be known to those in the art.

“Positive-negative selection” refers to the process-of selecting cells that carry a DNA insert integrated at a specific targeted location (positive selection) and also selecting against cells that carry a DNA insert integrated at a non-targeted chromosomal site (negative selection).

A “transgenic animal” refers to a genetically engineered animal or offspring of genetically engineered animals. A “chimeric embryo” is an embryo that has populations of cells with different genotypes. Thus, a transgenic animal or chimeric embryo usually contains material from at least one unrelated organism, such as from a virus plant, or other animal. The term “transgene” refers to a polynucleotide from one source that has been incorporated into genome of another organism. The transgene can be obtained from any source, for instance, isolated from a different organism, species or synthetically produced. The transgene can be a gene, gene fragment or multiple genes. Suitable sizes of transgenes can be determined by methods known in the art.

The present invention provides the first isolated population of rat embryonic stem cells. The invention also provides novel methods of deriving ES cells from non-mouse species. By employing novel culture conditions, the present inventor has derived isolated populations of rat ES cells from embryonic tissue. These novel methods are equally applicable to target species other than rats. Previous groups have unsuccessfully attempted to culture rat ES cells by amount and/or kind of growth factors and feeder cells added to the culture medium. No matter what the medium, previous rat cells cannot be maintained in culture without differentiating. These cultured rat cells are not transmitted into the germ lines of chimeric rats. It appears as though rat embryonic cells reach a critical stage in culture. At this stage growth factors and feeder cells are insufficient to promote the derivation of a undifferentiated, pluripotent ES line. The present invention overcomes this problem by co-culturing the rat embryonic cells with cells of a non-target ES cell line. Contact with the ES cell line appears to support the isolated embryonic cells through this crisis in culture. Once past this critical point, the rat ES cells will proliferate on their own and maintain their undifferentiated, pluripotent phenotype.

I. Sources of Pluripotent Embryonic Stem Cells

Using the methods described herein, ES cells can be isolated from any species. Examples described herein include ES cells derived from the inbred line of Long Evans (LE) rats, available from Simonsen (inbred for 16 generations) or from Sprague-Dawley rat strains. LE rats have appropriate coat color (black, hooded) for identifying chimeras when the LE ES cell candidates are injected into an albino strain. Large numbers of embryos have been obtained from this strain and these cells adapt well to tissue culture conditions. LE animals can also be time-mated by the supplier, reducing the size of the animal colony that must be maintained and the time involved in raising animals to breeding age. Unlike the mouse, strains of rat vary greatly in timing of embryonic implantation and each reacts differently to superovulation and delayed implantation procedures. To the extent that these variations affect the present invention, they can readily be determined by methods known in the art.

Non-mouse, target ES cells can be derived from any suitable cell. Non-limiting examples are blastocysts (non-delayed), blastocysts whose implantation has been experimentally delayed (delayed blastocysts), and primordial germ cells. PGCs can be isolated from various stages of embryonic development, for instance stage 13 embryos. For humans, cells obtained from spontaneous and elective abortions can be employed. Cells can also be obtained from embryos produced by in vitro fertilization techniques.

A. Blastocysts and Delayed Blastocysts

Blastocysts can be isolated by any method known in the art. For example, timed-mated females can be sacrificed on about day 4.5 after mating (day 0.5 is the morning after mating), and blastocysts are collected from the uteri by the method described for mice, for example in A. Bradley in TERATOCARCINOMAS AND EMBRYONIC STEM CELLS: A PRACTICAL APPROACH (E. J. Robertson, ed., 1987).

Preferably, timed-mated females are ovariectomized when embryos are in the oviducts, approximately day 3.5 after mating. After ovariectomy, animals receive daily injections of progesterone (5 mg/animal/day, subcutaneous injection), as described in Buchanan (1969) J. Reprod. Fert. 19:279-283. Between about six to about 11 days later, blastocysts that have not implanted are collected from the uterus using any method described in the art (e.g., A. Bradley, supra). Delayed blastocysts are usually larger than normal blastocysts, and lack the zona pellucida layer. Blastocysts and-delayed blastocysts may be cultured as described herein, preferably on feeder cells in individual wells of a 24-well plate.

Embryos produced by in vitro fertilization can be cultured to the blastocyst stage for the isolation of ICMs.

B. Primordial Germ Cells

Primordial germ cells can be also be isolated from embryos. Although the stage of the embryo tissue is not believed to be critical, in an embodiment directed to production of rat ES cells, timed mated females are sacrificed on approximately day 9.5 of pregnancy and embryos dissected from extraembryonic tissues. At this age, rat embryos are at stage 13, equivalent to the mouse on day 8.5. (stage is determined by somite number). The caudal region of the embryos, preferably from the last somite to the allantois, is dissociated into a single cell suspension with trypsin-(0.5%) and gentle trituration with a micropipette. Cells are plated, for instance into in 24-well dishes with or without feeder cells as described below. At this stage, there are approximately several hundred PGCs in each rat embryo.

II. Culture Conditions

The present invention employs culture conditions which promote ES cell derivation.

A. Cultures of Embryo Tissue

The primary blastocysts from which the ES cells are derived are grown in any appropriate medium under any conditions which allow for growth and proliferation of the ES cells. For instance, one suitable medium is mouse ES medium (DMEM with glutamine and high glucose (Gibco) supplemented with 15% fetal bovine serum (FBS: HyClone), 1 X non-essential amino acids, 0.1 mM 2-mercaptoethanol, and antibiotics).

Primary primordial germ cells (PGC) are preferably cultured in mouse ES medium containing exogenous growth factors, for example LIF, SCF and bFGF. Other growth factors which may be used still be known to those skilled in the art. The appropriate concentrations of the grouch factors can be readily determined by those skilled in the art. As described herein, 2000 U/mL LIF (Gibco); mouse SCF, 60 ng/mL; human bFGF, 20 ng/mL (Genzyme) have been shown to be effective. In addition, the cells can be cultured in ES cells prepared from other species, for example ES cells previously prepared as described herein.

Secondary and subsequent PGC cultures can be cultured in the same medium or a medium lacking exogenous growth factors. In normal and delayed blastocyst culture, the inner cell mass (ICM) is visible after about-three days of culture. The ICMs are removed under conditions that minimize contamination with other cell types, for example, after about 3 to 5 days in culture. In one embodiment, the ICM is removed using a micropipette and then dissociated with 0.25% trypsin. The ICM is dispersed either to a single cell suspension or more preferably, gently to produce small groups of cells. In one embodiment ICM cultures are cultured in 6 well dishes and colonies arising from the dispersed ICMs will be selected by morphological criteria. Exogenous growth factors (for example, bFGF, LIF, and SCF), alone or in combination, may be added to the cultures.

Optionally, the ICM cultures are cultured on a feeder layer, for instance mitotically inactivated SNL 76/7 cells, a feeder line that produces leukemia inhibitory factor (LIF) and stem cell factor (SCF). Feeder cells that induce differentiation (change in morphology, loss of alkaline phosphatase (AP) stain) should not be used.

B. Co-Cultures with ES Cells

A critical step in deriving embryonic stem cells is culturing the embryo tissue in the presence of pluripotent embryonic stem cells. Without being bound by one theory, it seems that the co-culture method is effective because of cell contact between ES cells and because of self-conditioning of the culture medium by ES cells. The present inventor noted that purified growth factors are not sufficient to provide optimal maintenance of pluripotence and proliferation of undifferentiated ES cells. For mouse ES cells to have a high probability of germ line transmission, mouse ES cells must be cultured on feeder layers, with serum. Most importantly, the inventor also noted that mouse ES cells differentiate much more easily and often when they were cultured at low density rather than high density. These observations led the present inventor to the claimed co-culturing methods.

As noted above, the embryo tissue can come from any source, preferably a non-mouse donor. In one embodiment, the embryo cells are ICMs from non-delayed or delayed blastocysts. In another embodiment they are primordial germ cells. In yet another embodiment, they are delayed blastocysts. At least one embryo tissue cell is used although between 1 and 50, preferably between about 5 and 10 single cells can also be combined into a single culture.

The selected embryo tissue cell(s) can be isolated and immediately co-cultured with the non-target embryonic stem cells. Preferably, the embryo tissue cells are cultured in vitro for a short time before adding the non-target embryonic stem cells to the culture, for instance for approximately 3 days. In a preferred embodiment, the co-culture is established before the embryo cells begin to differentiate. Optionally, the primary embryo tissue cultures can be cultured on a feeder layer of cells, for instance STO or SNL 76/7cells.

Any known pluripotent embryonic stem cells can be used in the co-culture condition. Although potentially as few as one ES cell may be required in co-culture, more may be required, for example between 10 and 100. It is preferable to use as few cells as required to derive target ES cells as too many added non-target ES cells may outcompete the generally slower dividing target species embryonic cells. Perferably, the non-target ES cells are mouse ES cells. Procedures for the isolation of mouse ES cells have been described (see, e.g., Martin, 1981; Ledermann et al., 1991 and Matsui et al., 1992).

In a preferred embodiment, the non-target embryonic stem cells are mouse ES cells having a negative selectable marker gene. Accordingly, the mouse ES cells can be selectively removed from the co-cultures. There are number of suitable selectable markers, including, for example HPRT and thymidine kinase. Other negative selection marker genes will be known to those skilled in the art.

In a preferred embodiment, the non-target mouse ES cells are a cell line that lacks HPRT, either by knock out or by, natural mutation. Suitable ES cell lines are incapable of reverting to HPRT-positive (HAT-resistant) phenotype. The co-cultured cells from the target species are HPRT-positive and survive HAT treatment. As described in the Examples below, control, containing only the HPRT-negative mouse cells, did not survive HAT treatment. In yet another embodiment, the non-target ES cells have been mitotically inactivated by gamma-irradiation.

Another means to identify the ES cells from the target species is to use, as the source of the co-culture, blastocysts from a cross between wild-type female rats and a male transgenic, wherein the transgene is a reporter gene. For example, female Sprague-Dawley rats can be crossed with a homozygous male carrying a randomly inserted, stably integrated LacZ reporter gene, under the control of a metallothionine promoter. The LacZ protein expression is thus inducible by metal ions such as zinc or cadmium. The blastocysts isolated from these crosses are then co-cultured with selectable non-target ES cells. After selectively killing the added non-target ES cells, the remaining cells can be induced to express LacZ in culture. As described in the Examples below, the claimed methods result in rat populations that are blue (i.e. express LacZ).

III. Characterization of ES Cells

The ES cells obtained using the methods claimed herein can also be assayed for ES cell phenotype. Typical cell surface markers expressed by ES cells include alkaline phosphatase and anti-SSEA-1. In vitro assays for differentiation, using embryoid body formation followed by culture to produce differentiated cell types, and retinoic acid induction of differentiation. The pluripotency of putative ES cells can also be demonstrated by showing the ability of subclones derived from isolated single cells to differentiate into a wide variety of cell types and by the formation of teratocarcinomas when injected into a whole animal. The cells can be assayed at any stage of the process.

A. Histochemistry

Pluripotent ES cells express specific cell surface markers which can be histochemically detected using antibodies or calorimetric (or enzymatic) assays. As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

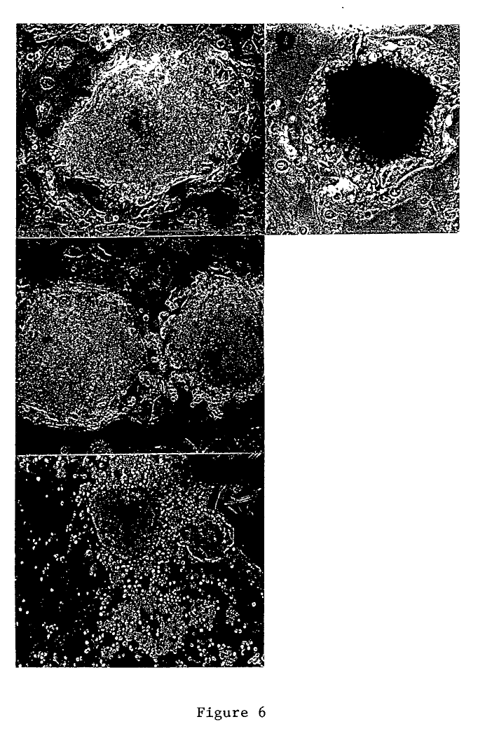

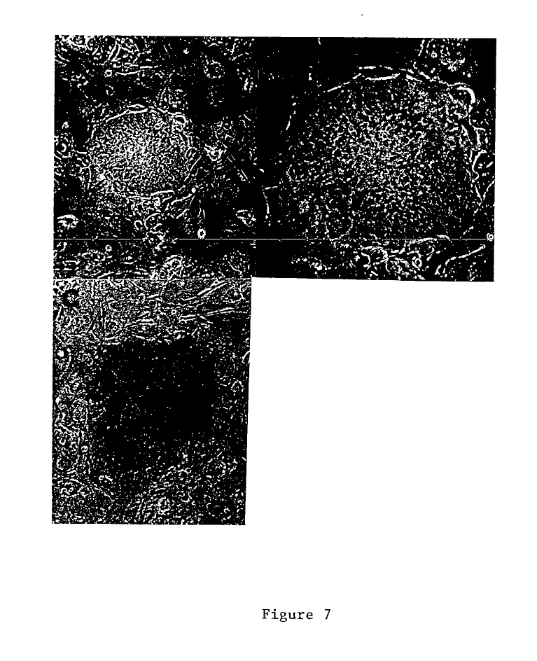

Monoclonal or polyclonal antibodies can be used to detect ES cell markers. The anti-SSEA-1 recognizes a glycolipid on the surface of undifferentiated mouse stem cells. Alkaline phosphatase (AP) activity is characteristic of primordial germ cell, blastocyst; inner cell mass and ES cells. FIGS. 6 and 7 show that rat ICM and PGCs co-cultured with mouse ES cells are AP positive (see FIGS. 6D and 7C). Both AP assays and SSEA-1 antibodies are commercially available.

B. Embryoid Bodies

Pluripotent ES cells can differentiate in culture into embryoid bodies containing multiple cell types. ES cells are multi-layered and the embryoid bodies which result from these multi-layered cells contain endodermal, mesodermal and ectodermal tissues and structures. In contrast, undifferentiated cells (non-ES) have one epithelial layer and develop embryoid bodies composed of a single cell type, epithelial cells. For example, in mouse ES cells, it has been demonstrated that removal of certain growth factors or treatment with inducing agent's such as retinoic acid or 3-methoxybenzamide is frequently accompanied by the generation of complex cystoid embryoid bodies in which endodermal, mesodermal and ectodermal formations can be detected. One easily observed indicator of pluripotence is formation from an embryoid body of cardiac tissue which spontaneously contracts in the culture dish. Generally, the ES cells described herein form embryoid bodies having a variety of cell types, including spontaneously contracting cardiac tissue. See, for example, Example 4, section B.2.

VI. Targeted Mutation of Genes Using ES Cells

In theory, any strain of a particular species could be used as a host blastocyst for injection of ES cells of that species. In practice, however, experiments with mouse ES cells have shown that some combinations do not work as well as others, and the choice of host can be critical to obtaining germ-line transmissions. When the rat ES cells are derived from the Long Evans strain, a preferred host is the Fischer 344 strain, since this albino strain would allow assessment of ES cell contribution from LE ES cells by observing pigmentation. Alternatively. ES cells derived from albino rat strains (e.g., Fischer 344) can be injected into pigmented host strains. The Fischer 344 albino animal lacks coat color markers to detect contribution of ES cells in a chimera when injected into an albino host blastocyst, so promising cell lines will be injected into blastocysts of a pigmented strain, for example, Long Evans or Dark Agouti. The coat color markers are a convenience rather than a necessity, since ES cell-derived cells could be detected by other means. There are two possible strategies for detecting cells: 1. endogenous isozyme or molecular markers and 2. introduced protein or genetic markers. Isozymes of GPI and other ubiquitous enzymes have been identified for many strains of rat and can be detected in blood samples or biopsies by established techniques see e.g., Robertson, supra)

Another technique that may be more generally useful is the introduction of molecular markers into the ES cells in culture. For example, a marked cell line can be created by introducing β-galactosidase or tyrosinase plasmids; β-galactosidase is detectable by staining, and has the advantage of allowing examination of chimeras as embryos. The tyrosinase gene would convert the albino cells to cells capable of producing pigment, allowing direct visualization of coat color chimerism. Methods for ES cell transfection are described herein and are known in the art, β-galactosidase and tyrosinase plasmids are commercially available. The advantage of this approach. is that any animal strain could be used for the blastocyst host, including the strain from which the ES cells originated.

The strength of ES cell technology is the ability to genetically modify cells in culture, analyze the cells for the correct modification, then produce a new line of animals from the cells. To exploit the powerful tool that ES cells provide, methods for introducing genetic modifications and for analyzing the ES cells have developed rapidly in the last several years. Gene targeting strategies allow inactivation of specific genes by homologous recombination. Selection techniques have been developed to improve identification of rare targeted events and to introduce subtle mutations. (see, e.g., Hasty et al. (1991) Nature 350:243-246; U.S. Pat. No. 5,629,159). Efficient methods for ES cell analysis, using microtiter plates and rapid DNA preparation techniques allow screening of thousands of clones for extremely rare events. In view of the non-mouse ES cells described herein, known methods of producing transgenic animals in mouse can readily be applied to other species.

ES cell technology has recently generated an important breakthrough in transgenic animal research. In three new reports, including one from the present inventor, ES cells were used for producing transgenic mice which express large genomic DNA fragments cloned in yeast artificial chromosomes (YACs). (See, e.g., Strauss et al. (1993) Science 259:1904-1907; Brinster (1988) Proc. Nat'l Acad. Sci. USA 85:836-40; Choi et al. (1993) 4(2):117-23 and 4(3):320). The rationale for this technique is the expectation that genomic transgenes which retain the content and organization of the locus are much more likely to be correctly expressed. However, many genes exceed the cloning capacity of conventional plasmids (less than 80 kb), and the traditional route to transgenesis, by microinjection of zygotes, may have an upper DNA size limitation because of the shear forces caused by forcing DNA through a microinjection pipette. The YAC has a cloning capacity of over 2000 kb. Transgenic rats via YAC transfection of rat ES cells can be created using known techniques. Virtually any gene can be disrupted using ES cells or transferred intact using YACs. In the rat, genes involved in neurodegenerative diseases (e.g., Alzheimer's disease, Parkinson's disease, Huntington's disease and the like) and cardiovascular diseases are particularly preferred.

VII. Use of ES Cells

A. Identification of Genes and Genetic Pathways Involved in AD

Human and rat ES cells can be made into neuroblasts that differentiate to neurons in culture, by manipulation of the culture conditions (e.g., adding retinoic acid, removing serum from the medium, changing culture substrata, adding specific growth or growth-inhibition factors, or ligands for cell surface receptors). The cells can be made into embryoid bodies, which enhances certain kinds of differentiation, then treated with other factors that enhance neuronal differentiation. To improve the yield of neurons, neuroblasts or neurons can be selected from other cell types by first transfecting the ES cells with a transgene in which a neuron- or neuroblast-specific promoter drives expression of a selectable marker, a novel cell surface macromolecule, or a reporter gene (such as Green Fluorescent Protein or LacZ). In the case of cells transfected with a selectable marker, addition of a compound to the culture medium that enables the cells that express the transgene, but not cells without the transgene, to survive and/or grow, allows the neurons to be collected. In the case of cells trnasfected with a novel cell surface macromolecule gene, expression of the novel cell surface macromolecule would serve to allow selection of neurons by a number of means including, but not limited to, antibody binding, adhesion to substrata and by providing a fluorescent tag. In the case of cells transfected with a reporter gene, the cells expressing the reporter gene can be collected by FACS. Neurons or neuroblasts can also be separated from other cells by density gradient centrifugation or by using another intrinsic property that distinguishes them from other cells.

A gene expression profile of the ES-derived cells can be made by mRNA detection methods including, but limited to, Northern blot analysis, RNAse protection, reverse-transcription PCR and cDNA expression arrays or microarrays of expressed sequence tags (ESTs), oligonucleotides or cDNA. Before such analysis, the mRNA can be first amplified, preferably by a linear amplication method. Genes that are expressed in neurons but not in their ES cell precursors are candidates for neuron-specific drug targets. Further analysis of candidate genes is done by performing genetic manipulations of the precursor ES cells. For example, genes known to be involved in AD, such as the preselinins, apolipoprotein E, amyloid precursor protein (APP), can be “knocked out” by gene targeting through homologous recombination. Since the ES cells are diploid, both copies of the gene can be modified by selecting for gene conversion events or by targeting both alleles. In another example, the same genes are modified in situ by “knock-in” methods such as the cre-lox procedure and the hit and run procedure. Modification of protein domains and “motifs”, as well as introduction of specific mutations can be done with these methods. In a further example of genetic manipulation of the ES cells, genes are added, as cDNA constructs or as large genomic transgenes (e.g., BACs and YACs from genomic libraries). Human genes are added to the rat cells, as well as to the human cells. The genes to be modified are not limited to AD-related genes, but could include any gene that is of interest.

A protein expression profile of the cells can be made using, for example, immunoblot analysis, ELISA, two-dimentional polyacrylamide gel electrophoretic analysis and histochemical analysis. Specific proteins that are of immediate interest with regard to AD are fragments of APP such as soluble APP and A-beta amyloid. Cells expressing these genes, for example, and other genes associated with AD can be used to identify drug candidates and to test hypotheses about the specific interactions of genes, signaling factors, and proteins in neurons and in AD. Such cells can provide a picture of gene expression and its control in neurons.

B. ES Cells In Testing Of Drug Candidates

Drugs that are designed to directly affect neurons can be tested in human and rat ES cell-derived neurons. The results have predictive value both for preclinical animal studies and for clinical trials. Drugs that work through other cell types can be tested by co-culturing rat or human ES cell derived neurons with glial cells, such as astroglia, microglia, and oligodendroglia. The cell types for co-culture are obtained, for example, as primary cultures, cell lines and by deriving them from the human ES cells. Toxicity of drugs can also determined by using similar cultures. The effects of drugs are assessed by a variety of assays including, but not limited to, biochemical, immunochemical, or gene expression assays. ES cells can be modified genetically to examine the effects of single nucleotide polymorphisms (SNPS) or larger genetic differences on drug efficacy and direct toxicity. The differentiated cells are included in models of the blood brain barrier (BBB), to test the effectiveness of drugs that must cross the BBB.

C. ES-Derived Cells as Transplants for the Brain

Among the potential uses of neurons and neuron-associated cells derived from human ES cells are repair of tissues damaged by neurodegenerative disease and injury. Among the current uses are striatal grafts of dopaminergic neurons for Parkinson's disease (PD), repair of brain regions damaged by ischemic stroke, and to bridge spinal cord lesions. Additional uses include transplantation of neurons into regions of the brain or body affected by: AD, peripheral neuropathy, ALS (amyotrophic lateral sclerosis), and other neurodegenerative diseases such as ataxias (trinucleotide-repeat diseases). The cells to be transplanted include neurons and neuron-supportive cells, such as glia or fibroblasts, or combinations of cells that support each other in a transplant.

Such cells for implant are genetically unchanged, or are modified to produce specific neurotransmitters, such as dopamine for PD, or specific growth factors to support themselves or other cells.

D. Additional Uses for ES Cells

To reduce the chance of graft rejection, the ES cells can be genetically modified to express different HLA types, to express molecules that mask the cells from the host immune system and/or by knocking out antigenic macromolecules.

The transgenic ES cells described herein can be used for in vitro screening or testing of compounds. In one aspect, the ES cells expressing genes involved in drug metabolism, such as the p450 gene, can be used in determining the effects of compounds on development and/or differentiation. Preferably, the ES cells express the human p450 gene.

The genetically modified ES cells described herein can be also used to create animal models of disease, useful in in vivo screening of potential therapeutics. Such animal models can include “humanized” rats (or other commonly used laboratory species). These “humanized” animals are created from ES cells which have been genetically modified to carry human genes associated with disease states. Such a generated model animal is useful for the post-discovery, pre-clinical phase of drug development.

The following examples are intended only to illustrate the present invention and should in no way be construed as limiting the subject invention.

### EXAMPLES

### Example 1

### Isolation and Culture of Rat Embryo Tissue

A. Rat Strains

A small colony of Brown Norway (BN; Charles River) rats of breeding age was established. Approximately 40 BN rats were housed in microisolator cages and replaced as animals were used in experiments. For development of the primordial germ cell procedures (see below), timed-mated Sprague Dawley rats (SD; Simonsen) were used. Timed-mated animals (F344 and Long-Evans) from a local supplier (Simonsen) were also used. The Long-Evans strain from Simonsen is a hooded rat that is about 50% black and 50% white. Although traditionally an outbred strain, Simonsen animals are effectively inbred, having been derived through 16 generations of brother-sister matings. For blastocyst injection, techniques were developed for producing large numbers of blastocysts from the Fischer 344 strain.

The F344 strain and the Simonsen LE rat produced large numbers of blastocysts and worked well for experimental procedures. The LE animals were not homogeneously pigmented, but provided the coat color markers required for assessment of chimeras. As a host strain for blastocyst injection, the AGUS strain and AB2 rats were chosen since they are nonhooded albino strains. Adult animals of these strains were not commercially available in sufficient numbers to use for the short term project. Instead, the methods for blastocyst injection were developed using F344 animals, for which an abundant source of adult pathogen-free animals (Simonsen) were available. In combination with LE ES cells, host blastocysts from the F344 strain should allow easy visualization of ES cell contribution in chimeras.

B. Production of Blastocysts From Natural Matings

Females mated for production of blastocysts were selected by estrus stage. To use the animals most efficiently, the vaginal plugs found on day 0.5 were checked for the presence of sperm, allowing monitoring of the fertility of the males. The timing of development in the BN strain was determined by flushing uteri and oviducts from a group of 14 pregnant animals at days 3.5, 4.5, and 5.5 after mating. It was determined that, unlike the mouse, development of BN rat embryos in individual animals varied by as much as a day. In general, early cleavage stage embryos were found in the oviducts on days 3.5 and 4.5, and morulae and blastocysts were in the uterus on days 4.5 and 5.5, but in individual animals embryos were found that ranged from zygote to blastocyst stag, particularly with the F344 and LE rat strains. Animals of these strains could be time-mated by the supplier (Simonsen) and shipped on the 3rd day after mating. Blastocysts could be collected from about 50% of the F344 rats and about 80% of the LE rats on day 4.5.

C. Superovulation of Rats

In order to superovulate rats, a method developed by Armstrong and Opavsky (1988) Biol. Reprod. 39:511-518 was adapted. Young female animals were implanted with osmotic minipumps, (Alza Corporation) containing porcine follicle stimulating hormone (FSH); the pumps were removed after 3 days and the animals mated. This method provided more predictable production of blastocysts in our colony, and may be used for production of host blastocysts for injection of ES cell candidates if natural matings of animals fail to provide sufficient blastocysts.

D. Delayed Blastocysts

Embryonic stem cell lines from delayed blastocysts of two strains of mouse had been previously derived. In the mouse, simple removal of the ovaries at day 2.5, when embryos have traveled down the oviducts, prevents implantation after blastocyst development. The procedure had to be considerably modified for use in the rat. The original procedure did not work in the rat, and no delayed blastocysts were obtained from BN animals ovariectomized on day 2.5 or 3.5, and examined for embryos 3 to 6 days later. No embryos were obtained from 11 Sprague Dawley animals implanted after ovariectomy with minipumps containing Depo-Provera. a progesterone analog used for delaying implantation of mouse blastocysts. The successful method combined ovariectomy on day 3.5 with daily injections of progesterone (5 mg/animal/day, subcutaneous injection). The method appears to be especially effective for Long-Evans rats.

E. Primordial Germ Cells

It was recently reported that cells having all of the in vitro properties of embryonic stem cells could be derived from primordial germ cells of 8.5 day (Stage 13) mice. Rat embryos of the same stage (at 9.5 days) were obtained from BN, SD, F344, and LE rats, dissected and dissociated the caudal portion (posterior to the last somite) and cultured them in medium containing basic FGF, stem cell factor and LIF (Matsui et al., 1992) on SNL76/7 cell feeder layers (see culture conditions, below).

### Example 2

### Culture Medium for Rat Cells

The culture medium used in most experiments was based on mouse ES cell medium, and contained high glucose DMEM (Gibco) supplemented with 15% fetal bovine serum (Hyclone), 1X nonessential amino acids (Gibco), and 2-mercaptoethanol (0.1 mM). Conditioned medium was prepared from Buffalo Rat Liver cells (BRL: ATCC), mouse AB-1 ES cells (provided by A. Bradley), and a rat blastocyst-derived cell line we derived (BNRB-1). Medium was conditioned by 2 days of culture with the cell lines, then filtered and frozen for future use. For experiments, the conditioned media were used at 50% mixed with fresh medium. Exogenous growth factors used were leukemia inhibitory factor (LIF: 1000-2000 U/mL; Gibco/BRL), basic fibroblast growth factor (BFGF: 20 ng/mL; Genzyme), and stem cell factor (SCF: 60 ng/mL; Genzyme).

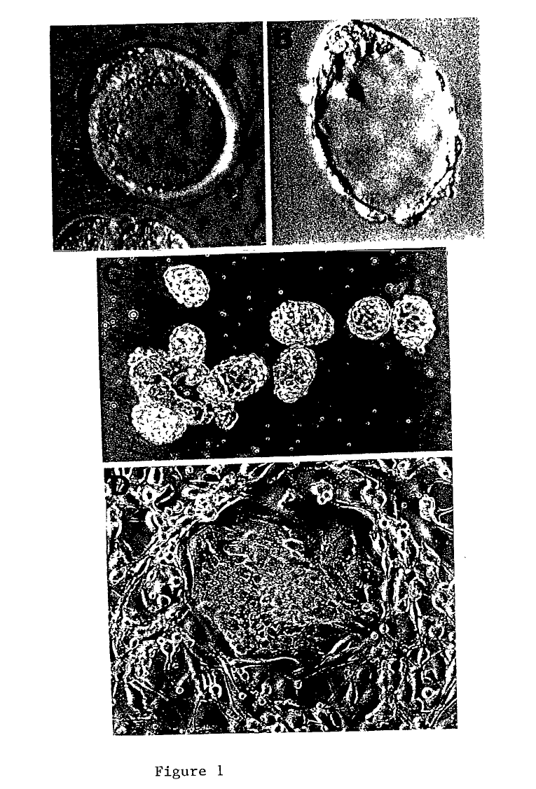

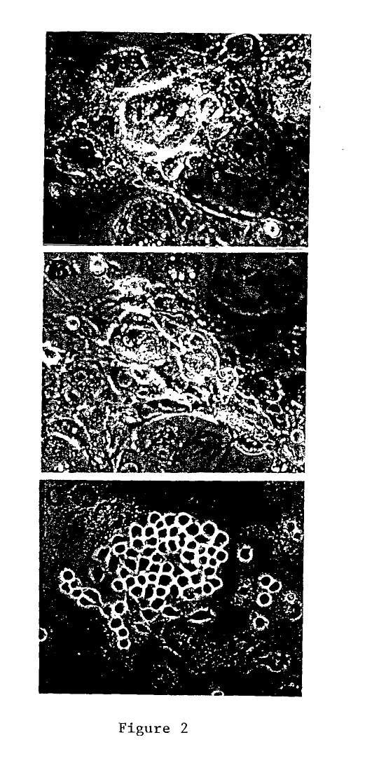

Normal and delayed blastocysts (shown in FIG. 1) were placed on fibroblast feeder layers made from LIF-producing mouse fibroblasts (SNL76/7) or embryonic rat fibroblasts and cultured for 3 to 7 days. All of the blastocysts attached and in almost all the inner cell mass (ICM) was visible (FIG. 1d). Culture medium (LIF; LIF and SCF, or LIF, SCF, and bFGF) made no apparent difference in the early blastocyst culture. The center mass of cells was usually removed from each culture 3-5 days after blastocyst culture, dissociated and subcultured on the same feeder type in the same medium. The secondary cultures always contained colonies of various morphology, including some that resemble the compact colony morphology of mouse ES cells (FIG. 2). After about a week in secondary culture, colonies that resembled ES cells were dissociated and subcultured. The subcultured cells were passed once and then frozen. Cells lines were derived from a non-delayed Brown Norway blastocyst (BNRB-1), a Fischer 344 delayed blastocyst (FRDB-1) and a Long-Evans strain.

Two types of feeder layers were used for these experiments. SNL76/7 is a mouse fibroblast line used as feeder layers for mouse AB-1 ES cells (Soriano et al. 1991). SNL76/7 cells are neomycin resistant and produce LIF and stem cell factor. Rat embryo fibroblasts (REF) were prepared from rat embryos by a method used for mouse embryos and described, for example, in Doetschman et al. (1985) J. Embryol Exp. Morph. 87:27-45. The feeder cells were mitotically inactivated by treatment with mitomycin C. Mitomycin C sas toxic to the REF cells at the 10 μg/ml concentration used for SNL cells, so the concentration was reduced to 4 μg/ml, and the cells were treated for a shorter time. Cells were cultured on feeder layers in 6-well or 24-well tissue culture plates, glass coverslips, or glass culture slides (LabTek). Alternatively, feeder layers can be mitotically inactivated with gamma-irradiation (see, Robertson, supra).

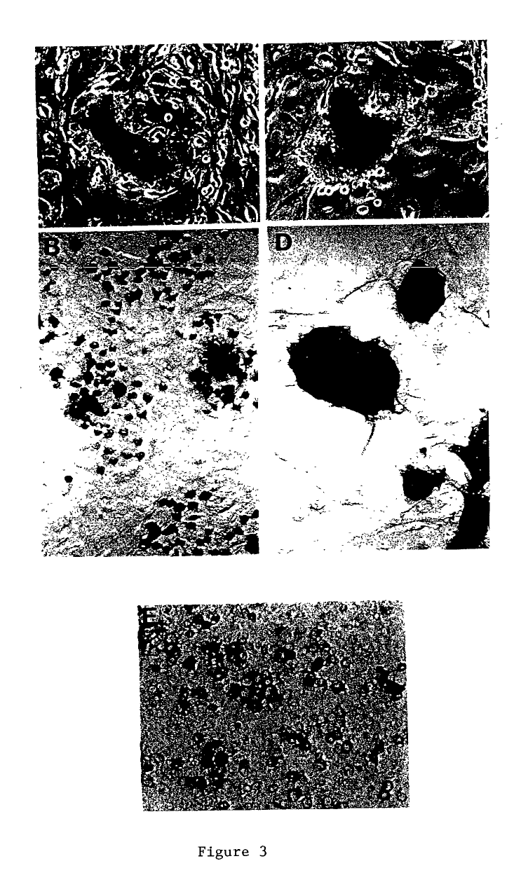

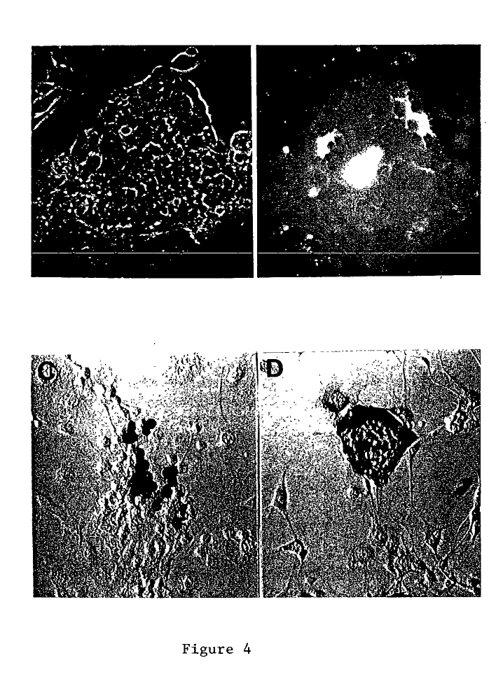

Primordial germ cells were cultured from tissues obtained from dissections of 9.5 day embryonic rats from Sprague-Dawley and Long-Evans (LE) strains using the methods reported for mouse, and stained the cultures for AP as described below. Several of the cultures contained colonies of cells that stained with AP (FIG. 3e), and are therefore likely to be PGCs.

A. Effect of Culture Conditions on Rat Blastocyst-Derived Cells

In a preliminary experiment, a variety of conditioned media and exogenous growth factors were tested in a search for conditions that might enhance the growth or maintenance of cells with embryonic stem cell characteristics. The results are summarized in Table 1. The extent of AP staining was estimated for cultures shown in FIG. 6. Medium conditioned by BRL cells had no noticeable effect on AP staining in the rat cultures. BRL cells produce LIF, SCF, and other factors that maintain mouse ES cells in an undifferentiated state. Similarly, mouse ES cell conditioned medium also had no effect on the rat cells. Medium conditioned by high density BNRB-1 cells appeared to decrease the AP stain in cultures of the same cell line, suggesting that the cells may produce factors that cause them to differentiate or inhibit proliferation of undifferentiated cells. Of the conditions so far tested, only exogenous bFGF appeared to have a qualitative effect of increasing the number of AP-positive cells.

TABLE 1
Effect of culture conditions on rat blastocyst-derived (BNRB-1) cells

| | | Alkaline |
|---|---|---|
| | | phosphatase |
| Culture medium | Feeder layer | stain^{a} |
| Control medium | SNL76/7 | ++ |
| QNRB-1 conditioned medium (50%) | SNL76/7 | −/+ |
| BRL conditioned medium (50%) | SNL76/7 | ++ |
| Mouse ES cell conditioned medium (50%) | SNL76/7 | + |
| BRL c.m. + BNRB-1 c.m. (25% + 251/*) | SNL76/7 | + |
| Control medium + 20 ng/ml bFGF | SNL76/7 | +++ |
| Control medium | Rat fibroblasts | −/+ |
| Control medium + 20 ng/ml bFGF | Rat fibroblasts | −/+ |

^{a}Extent of staining was estimated from cultures shown in FIG. 6. Scores indicate the relative amount of staining in the culture. −/+ indicates that very little staining was detectable.

### Example 3

### Derivation of Rat ES Cells in Co-Culture

Pluripotent ES cells were derived by co-culturing the rat cell lines described above with mouse ES cells. Rat ICMs for non-delayed blastocysts were cultured for 3 days on STO feeder layers. After 3 days, approximately 20-50 cells were present in the ICMs. Before differentiation began (usually at about 4 days), the rat ICMs were mixed with a mouse ES-cell line called Del 19.2. This line, which has a “knock-out” of 19.2 kb in the single (X-linked) copy of the HPRT gene, was obtained from Dr. Allan Bradley. Del 19.2 cells are HPRT− do not survive in HAT medium and are incapable of reverting to a HPRT+ phenotype. The rat ICM:mouse ES cell co-cultures were passed when confluent at least two times, with trypsin. After between about 5 days and 2 weeks, HAT medium was added to kill the mouse Del 19.2 cells. The rat cells survive HAT treatment because they are HPRT-positive. Hundreds of surviving colonies were obtained. As described in detail below, these colonies maintained markers of pluripotent ES cells.

### Example 4

### Characterization of Rat Cells Before and After Co-Culture

Rat cells were characterized by various methods both before and after co-culture with ES cells. One cell type dominated in the first cell line derived (BNRB-1) in the absence of co-culture. These cells were round, retractile, and had a loose attachment to the substratum (FIG. 2C). Unlike mouse ES cells, these cells had little tendency to aggregate into tight clusters (compare FIG. 3B and 3D). This cell line was examined for the presence of two markers that are characteristic of mouse ES cells, alkaline phosphatase activity and the stage-specific embryonic antigen, SSEA-1. The second cell line (FRDB-1) forms colonies with a more cohesive morphology (FIGS. 2B, 2C), and has been analyzed for certain markers.

A. Histochemistry

Cells were fixed with 4% paraformaldehyde in PBS for AP or antibody stain. To detect alkaline phosphatase activity the cells were stained with a Vectastain AP reagent kit (Vector Labs) which produces a black reaction product in the presence of AP. Specificity of the stain was confirmed by levamisole inhibition. Anti-SSEA hybridoma supernatant (Developmental Studies Hybridoma Bank) was used at a 1:50 to 1:100 dilution and the antibody binding was detected with fluorescein or conjugated anti-mouse IgM (Vector labs). Cells grown on glass coverslips were viewed with Nomarski optics (Nikon Diaphot) or fluorescence (Leitz Laborlux); cells in tissue culture dishes were photographed with phase or brightfield optics. For a quantitative assay of AP-positive cells, cells were trypsinized, fixed with 2% paraformaldehyde, and stained in suspension with the AP reagent kit, and counted in a hemocytometer.

1. Alkaline Phosphatase

Alkaline phosphatase (AP) activity is characteristic of primordial germ cells, blastocyst inner cell mass, and ES cells of mouse. The inner cell mass rat blastocysts has AP activity (FIG. 3A) as does the ICM of mouse cultured under the same conditions (FIG. 3C). AP activity was consistently observed in cultures of BNRB-1 cells (FIG. 3B), but only in a small proportion of the cells. The staining was inhibited by levamisole (Vector Labs), which also inhibited AP activity in the control mouse ES cells.

A quantitative assay was developed to provide a clear measure of the proportion of AP-stained cells. As shown in Table 2, after multiple passages the proportion of AP-positive cells in the BNRB-1 line was only 5%. The FRDB-1 line was approximately 50% positive. Mouse ES cell cultures (AB-1) Were more than 90% positive. Rat cells were analyzed after co-culture by staining the colonies in the culture dish. The proportion of AP positive cells appeared to be almost 100% (see FIGS. 6 and 7).

TABLE 2
Alkaline phosphatase-positive cells in cell lines prior io co-culture.

| Cell line | Passage # | AP positive |
|---|---|---|
| BNRB-1 | 5 | 5% (6/140) |
| FRDB-1 | 5 | 48% (91/191) |
| Mouse ES cell | 12 | 92% (66/72) |

B. Pluripotence of Rat Cells

An important in vitro indicator of cell pluripotence is a demonstration that these cells form embryoid bodies composed of multiple cell types.

1. Embryoid Bodies from Rat Cells Before Co-Culture

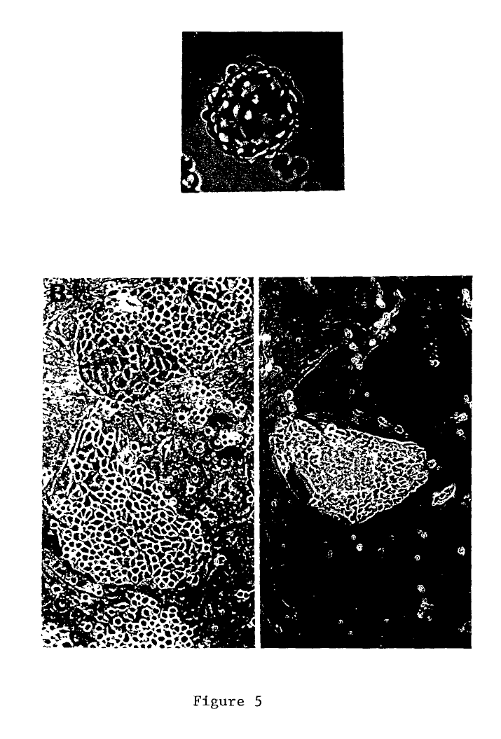

The BNRB-1 cells prior to co-culture showed an ability to differentiate in vitro, but they did not show the extent of pluripotence of mouse ES cells. When cultured under conditions that promote differentiation of mouse ES cells into embryoid bodies, the rat cells formed aggregates that developed into cyst-like structures (FIG. 5A). These structures resemble the simple embryoid bodies bounded by endoderm that form initially by mouse ES cells; unlike the mouse cells, however, the rat embryoid cysts did not continue to differentiate into more complex structures. In most cases the cysts appeared to be bounded by a single epithelial layer, but some developed multiple layers. When replated onto feeder layers, mouse embryoid bodies differentiate into a wide variety of cell types; the rat embryoid cysts failed to differentiate further when replated. This limited differentiation is consistent with idea that the dominant cell type in the population is differentiated endodermal cells.

Under certain culture conditions some BNRB-1 cell colonies underwent morphological differentiation. On rat embryo fibroblast feeder layers, mouse ES cells lost their AP stain and differentiated into a morphologically distinct cell type that resembled epithelial cells (FIG. 5C). Similarly, some of the rat cells on rat embryo fibroblast feeder layers showed less staining for AP and formed-colonies that resembled epithelial cells (FIG. 5B). Similar results were seen when the rat cells were cultured in retinoic acid, which induces differentiation in mouse ES lines (not shown).

2. Embryoid Bodies Formed After Co-Culture

After co-culture, the isolated and resuspended rat ES cells differentiated into a number of morphologically different cell types, including a “beating” mass of heart tissue. The ability to form beating heart and other embryoid bodies is indicative of pluripotent ES cells.

These cells are then implanted into an immune deficient host animal (e.g., nude rats or nude mice) to determine whether they formed teratomas. The cells are subcloned and karyotyped and are injected into host blastocysts.

### Example 5

### Derivation of Rat ES Cells in Co-Culture

Sixteen blastocysts were obtained from PVG rats at day 5 after mating and plated into a single organ culture dish in 1 ml of ES cell medium (same as described earlier but with 20% fetal bovine serum) containing 2000U of mouse LIF. The dish contained a mitotically inactivated STO cell feeder layer. Blastocysts were cultured for 3 days, when clusters of ICM cells were evident. The ICM cells were removed from the dish with a glass pipette, and incubated for 30 min. in a solution of calcium and magnesium-free PBS containing 1 mM EGTA. The ICMs, which dissociated into small clumps of cells, were placed in a 6-well culture dish on a feeder layer in ES medium (without LIF). To the same well were added 1.5×10⁵ mouse ES cells (HPRT-Del 19.2 cells). A control well contained an equal number of mouse ES cells but no rat cells.

After 3 days, the cells in both wells were dissociated with trypsin (0.25% in 1 mM EDTA; 15 min, 3° C.). Nine-tenths of the well in which the rat ICMs were plated was frozen by standard cell preservation methods, and the remaining tenth was plated into a new 6 well chamber with feeders, using the same medium. Similarly, the control well was passaged at a dilution of 1 to 10. After three more similar passages and cell freezing at 3 day intervals (the rat cell-containing culture was split at the second passage to generate two wells), both types of cultures were trypsinized and replated (without dilution) into HAT-containing ES cell medium on feeder layers. Thereafter the medium (containing HAT) was changed every day for three days. The media changes were made often because the products of dying cells can often damage surviving cells in the same dish. On the third day, the cultures were examined carefully with a microscope. The control culture contained STO feeder cells, but no visible embryonic stem cells, indicating that all of the mouse HPRT- cells had been killed by the HAT medium.

The experimental culture, originally containing rat ICMs, contained numerous colonies of cells. One of the 6-wells (“A”) contained 38 colonies containing an estimated 100-500 cells; of these, three appeared to consist entirely of the differentiated cell type that has been previously described as “endoderm”, six colonies consisted of mixtures of cellular morphologies, and the remaining 29 consisted solely of cells that looked indistinguishable from mouse ES cells at the microscopic level. Well “B” contained 30 colonies; 7 were “endodermal”, three were mixed, and 20 consisted of ES-like cells. The colonies were stained for AP, and all colonies containing ES-like cells were positive for AP, showing that the rat cells not only looked like mouse ES cells, but also exhibited markers that identify mouse cells as ES cells (FIG. 7).

To be certain that all mouse cells had been killed in the control culture, the medium in that dish was exchanged for ES medium without HAT, and the culture was maintained for 10 more days with daily medium changes. No ES cells appeared even after 10 days, demonstrating the clearance of all of the mouse ES cells by HAT treatment.

### Example 6

### Transgenic Rat Generation Using Rat ES Cells

Rat ES cells isolated as described are tested for pluripotence in vivo by blastocyst injection. Long Evans rat ES cell are injected into host blastocysts from the albino Lewis strain. The blastocysts are allowed to develop to term in a surrogate mother and the pup examined. The pup has patches of brown coat and eye color, indicating contribution from the Long Evans ES cells.

As is apparent to one of skill in the art, various modification and variations of the above embodiments can be made without departing from the spirit and scope of this invention. These modifications and variations are within the scope of this invention.

1. An isolated population of non-mouse embryonic stem cells.

2. The population of cells according to claim 1 wherein the cells are rat.

3. A method of obtaining embryonic stem cells from a target species, the method comprising:
(a) co-culturing cells obtained from an embryo of the target species with non-target embryonic stem cells under conditions which favor growth of the embryonic stem (ES) cells from the target species; and
(b) isolating the ES cells from the target species.

4. The method according to claim 3 wherein the non-target embryonic stem cells of step (a) are mouse.

5. The method according to claim 3 wherein the cells obtained from an embryo of the target species of step (a) are derived from inner cell masses (ICMs).

6. The method according to claim 3 wherein the cells obtained from an embryo of the target species of step (a) are primordial germ cells (PGC's).

7. The method according to claim 3 wherein the target species is non-mouse.

8. The method according to claim 7, wherein the target species is selected from the group consisting of rat, sheep, bovine, and human.

9. The method according to claim 8 wherein the target species is rat.

10. The method according to claim 3 wherein the non-target embryonic stem cells of step (a) lack a positive selection marker.

11. The method according to claim 10 wherein the positive selection marker is selected from the group consisting of an antibiotic resistance gene or an HPRT resistance (HPRT) gene.

12. The method according to claim 11 wherein the positive selection marker is an HPRT gene.

13. The method according to claim 3 wherein the non-target embryonic stem cells of step (a) carry a negative selection marker.

14. The method according to claim 13 wherein the negative selection marker is HPRT or herpes simplex virus thymidine kinase (HSV-tk).

15. The method according to claim 3 wherein the embryo cells from the target species are cultured on a feeder layer of cells.

16. The method according to claim 15 wherein the feeder layer of cells is SNL 76/7.

17. The method according to claim 3 wherein the non-target embryonic stem cells are mitotically inactivated.

18. A genetically modified non-mouse ES cell.

19. The genetically modified ES cell of claim 18 wherein the cell is rat.

20. The genetically modified ES cell of claim 18 comprising one or more transgenes.

21. A chimeric embryo containing the isolated population of non-mouse ES cells of claim 1.

22. The chimeric embryo according to claim 21 wherein the ES cells are rat.

23. A chimeric embryo containing a genetically modified non-mouse ES cell prepared according to the method of claim 3.

24. The chimeric embryo according to claim 23 wherein the function of one or more genes is disrupted.

25. The chimeric embryo according to claim 23 wherein the non-mouse ES cell is rat.

26. The chimeric embryo according to claim 23 wherein the genetically modified non-mouse ES cells include one or more transgenes.

27. An animal containing cells arising from the isolated population of non-mouse ES cells of claim 1.

28. The animal according to claim 27 wherein the non-mouse ES cells are rat.

29. An animal containing cells arising from a genetically modified non-mouse ES cell.

30. The animal according to claim 29 wherein the non-mouse cell is rat.

31. The animal according to claim 29 wherein the genetically modified non-mouse ES cells include one or more transgenes.

32. The animal according to claim 29 wherein the function of one or more genes is disrupted.
